(19) United States
(12) Patent Application Publication
(10) Pub. No.: US 2002/0068908 A1
(43) Pub. Date: Jun. 6, 2002

(54) Cap for a syringe

(76) Inventor: Wu-Chung Sun, Kaohsiung (TW)

Correspondence Address: Jackson Walker L.L.P., Suite 2100, 112 E. Pecan Street, San Antonio, TX, 78205, US

(21) Appl. No.: 09/728,259

(22) Filed: Dec. 1, 2000

(51) Int. Cl. A61M 5/32

(52) U.S. Cl. 604/192

(57) ABSTRACT

A cap has a tubular body and a base formed on one end of the tubular body. The cap can be stably stood on a flat object, such that the cap does not need to be held when the needle is inserted into the cap. The user does not need to hold the cap. The user will not be punctured. The safety of disposing of the used syringe can be improved. In addition, the needle can be punched out of the hub and drop through the opening in the cap. The plastic cap and hub does not need to be melted and burned with the needle. The generation of hazardous gas is avoided.

### BACKGROUND OF THE INVENTION

1. Field of the Invention

The present invention relates to a cap, and more particularly to a safety cap for a syringe.

2. Description of Related Art

A plastic cap is always mounted on the hollow barrel of a syringe to cover the needle to keep people from being punctured by the used needle. However, because the user must hold the conventional cap when he or she puts the cap onto the hollow barrel, the user is easily punctured by the needle. In addition, the used needle must be destroyed by melting or burning to prevent the spread of disease. However, hazardous gas will form when the needle with the plastic cap and needle hub is melted or burned.

In another conventional embodiment, a safety device is formed with the syringe to prevent the user from being punctured. Clamps are formed on the needle hub and the plunger of the syringe. When the plunger is fully pushed into the hollow barrel of the syringe, the clamps will engage with each other. The needle hub with the needle will release from and be pulled fully into the barrel when the plunger is pulled back. The used needle will be fully enclosed and held in the hollow barrel to keep the needle from touching or puncturing anyone.

However, a specific mold is required to form the clamps. The cost for manufacturing the syringe with the conventional safety device is expensive. In addition, because the entire syringe including the hollow barrel, plunger and the hub must be melted or burned with the needle, hazardous gas is still generated.

To overcome the shortcomings, the present invention intends to provide a cap for a syringe to mitigate or obviate the aforementioned problems. I

### SUMMARY OF THE INVENTION

The main objective of the invention is to provide a cap able to stably stand on a flat object, such that the cap does not need to be held when the the needle is inserted into the cap. The cap has a tubular body and a base formed on one end of the tubular body. With such a cap, the needle can be inserted into the tubular body when the tubular body is standing on the base on a flat object. The user does not need to hold the cap so that the user will not be punctured. The safety of dealing with the used syringe can be improved.

The other objective of the invention is to provide the cap with the tubular body and the base. The tubular body has two openings. The needle can be punched out of the hub and the opening of the cap. The plastic cap and hub does not need to be melted and burned with the needle. The formation of hazardous gas is avoided.

Other objects, advantages and novel features of the invention will become more apparent from the following detailed description when taken in conjunction with the accompanying drawings.

### BRIEF DESCRIPTION OF THE DRAWINGS

FIG. 1 is a perspective view of a cap in accordance with the present invention;

FIG. 2 is a cross sectional side plan view of the cap in FIG. 1;

FIG. 3 is an operational side plan view in partial section of the cap in FIG. 1 with the needle of the syringe being inserted into the cap standing on a flat object; and

FIG. 4 is an operational side plan view in partial section of the cap in FIG. 1 with the needle being punched out of the hub and the cap.

### DETAILED DESCRIPTION OF PREFERRED EMBODIMENT

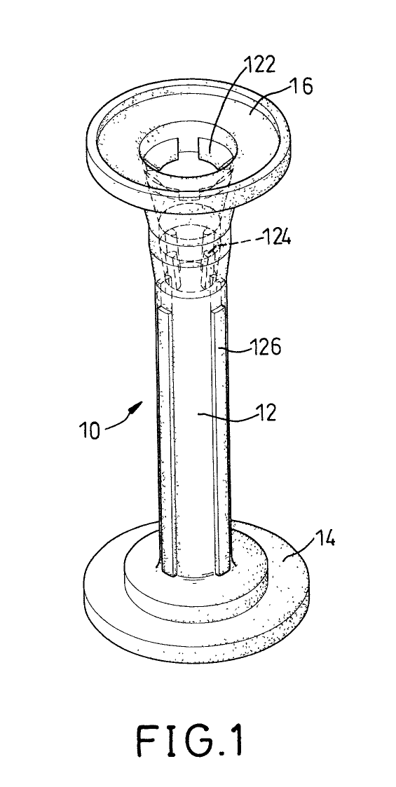

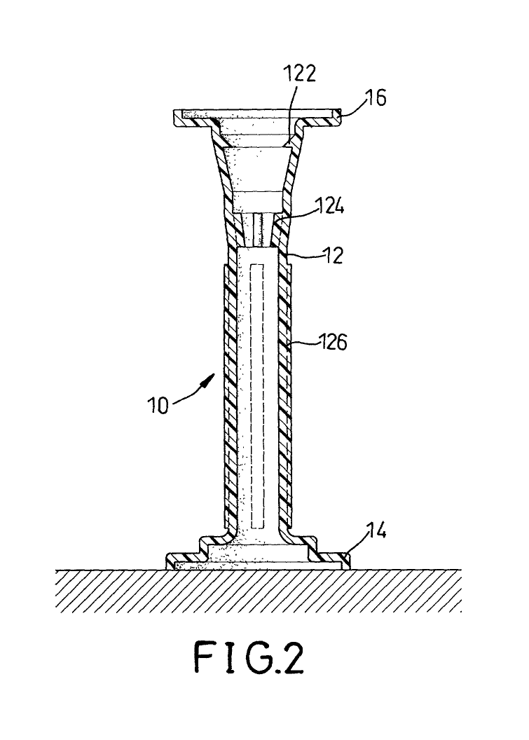

With reference to FIGS. 1 and 2, a cap (10) in accordance with the present invention comprises a tubular body (12) and a base (14). The tubular body (12) has an opening defined in each end of the tubular body (12). The base (14) extends out radially from one end of the body (12) with a desired area. In practice, the base (14) is a circle. The cap (10) is able to stand upright on the base (14) on a flat object like a table.

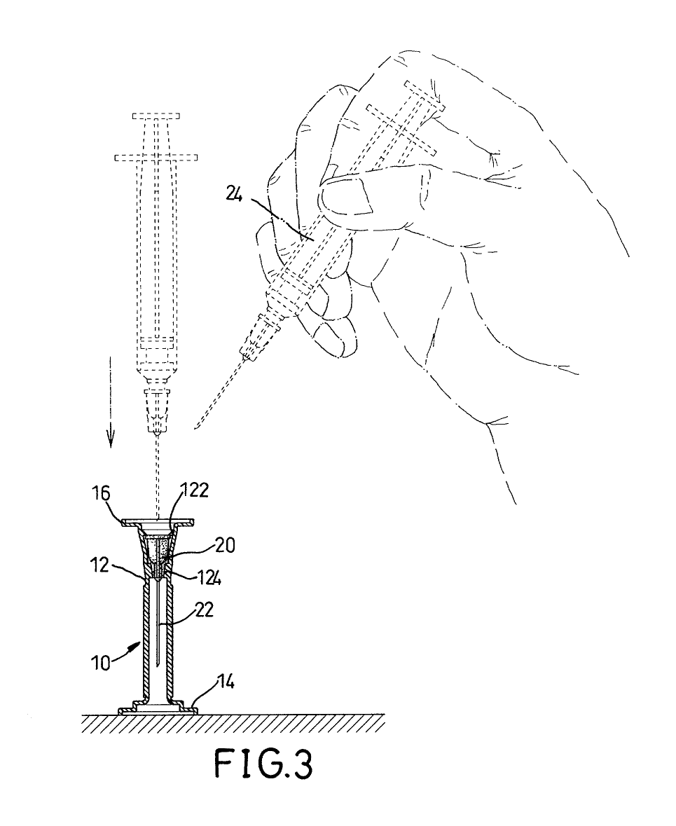

With reference to FIGS. 2 and 3, the uses can insert the used needle (22) and the needle hub (20) of a syringe into the cap (10) standing on a table. Consequently, the used needle (22) is shielded by the cap (10), such that the used needle (22) will not puncture anyone. Because the user does not need to hold the cap (10) when the needle (22) is inserted into the cap (10), the user will not be punctured by the needle (22). The safety of disposing of used needles is improved by using the cap (10).

Multiple tabs (122) extend into the inside of the cap (10) from the inner periphery of the tubular body (12) at the end opposite the base (14). When the needle hub (20) is inserted into the cap (10), the tabs (122) will clamp the needle hub (20) to keep the hub (20) from escaping from the cap (10). This prevents the repeated use of the needle (22). A hood (16) extends radially out from the tubular body (12) at the end opposite to the base (14). The hood (16) serves as a guide when the needle (22) is inserted into the cap (10).

Generally, the needle hub (20) of the disposable syringe can be disengaged from the hollow barrel (24). The needle hub (20) with the needle (22) is disengaged from the hollow barrel (24) after they are inserted into the cap (10). Multiple ribs (124) are formed on the inner periphery of the tubular body (12) to engage with the needle hub (20). When the user turns the cap (10), the needle hub (20) can be released from the hollow barrel (24) by the engagement between the ribs (124) and the needle hub (20). Multiple protrusions (126) are integrally formed on the outer periphery of the tubular body (12), such that the cap (10) is easily gripped and rotated.

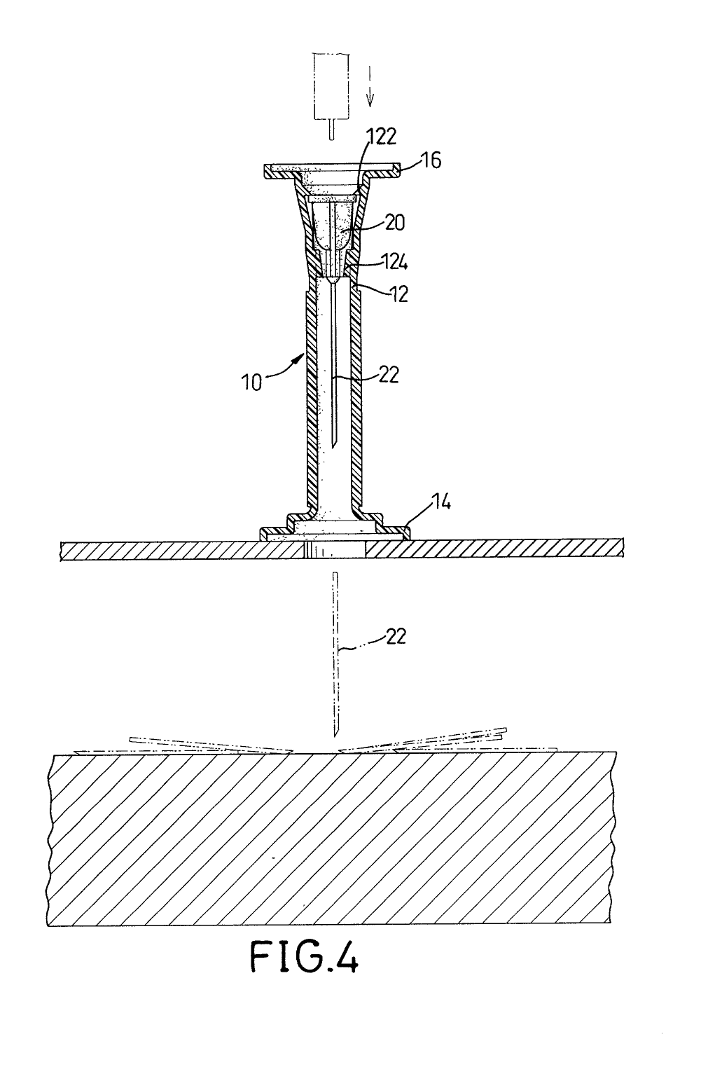

With reference FIG. 4, the cap (10) with the needle hub (20) and the needle (22) in the cap (10) is put into a punching machine, and the needle (22) is then punched out of the needle hub (20) and drops through the bottom opening in the tubular body (12). Consequently, the used needles (22) can be destroyed by melting or burning. The plastic cap (10) and the needle hub (20) can be recycled to manufacture other plastic products. No hazardous gas will be generated.

Even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A cap for a syringe having a hollow barrel, a needle hub detachably mounted on one end of the hollow barrel, a needle mounted on the needle hub and a plunger inserted into the other end of the hollow barrel, the cap comprising:
a tubular body with an opening defined in each end of the body to be adapted to receive the needle hub and the needle; and
a base extending radially out from one end of the body.

2. The cap as claimed in claim 1, wherein multiple tabs are formed on an inner periphery of the end of the body opposite to the base to be adapted to abut the needle hub.

3. The cap as claimed in claim 1, wherein a hood extends radially out from the body at the end opposite to the base.

4. The cap as claimed in claim 1, wherein multiple ribs are integrally formed on an inner periphery of the body to be adapted to be engaged with the needle hub.

5. The cap as claimed in claim 1, wherein multiple protrusions are integrally formed on an outer periphery of the body.
